(19) United States
(12) Patent Application Publication
(10) Pub. No.: US 2019/0246890 A1
(43) Pub. Date: Aug. 15, 2019

(54) Systems And Methods For Neuro-Ophthalmology Assessments in Virtual Reality

(71) Applicants: Harry Kerasidis, Dunkirk, MD (US); Gerald Howard Simmons, Sugar Land, TX (US); Chad Michael Watkins, Ashburn, MD (US)

(72) Inventors: Harry Kerasidis, Dunkirk, MD (US); Gerald Howard Simmons, Sugar Land, TX (US); Chad Michael Watkins, Ashburn, MD (US)

(21) Appl. No.: 16/274,233

(22) Filed: Feb. 12, 2019

Related U.S. Application Data
(60) Provisional application No. 62/629,353, filed on Feb. 12, 2018.

(51) Int. Cl. A61B 3/00; G02B 27/00; G02B 27/01

(52) U.S. Cl. CPC A61B 3/0041; A61B 3/0008; G02B 27/017; G02B 27/0093

(57) ABSTRACT

Neuro-ophthalmology, vestibular, ocular and oculomotor assessment systems using both unmodified and modified off-the-shelf virtual reality and mobile computing device connected and configured for those assessments, resulting in significant cost reductions per system. Unmodified systems using mobile computing device sensor data and user response for the assessments, and modified systems having electrooculogram electrode and/or photo sensors for electrooculogram signal recording, electrooculogram analysis synchronized with the virtual reality display, and additional modified systems for precise eye tracking. Additionally, specific methods of use for each system, and combinations of them, to assess balance, convergence, visual fields deficits, extra-ocular movement, tracking and targeting, and vestibulo-ocular reflex, among others.

### CROSS-REFERENCES TO RELATED APPLICATIONS

The present application claims priority benefit of U.S. provisional application No. 62/629,352 filed Feb. 12, 2018.

### FIELD AND BACKGROUND OF THE INVENTION

The present disclosure relates generally to systems and methods for vestibular and oculomotor neuro-ophthalmology assessments in virtual reality (VR). More particularly, embodiments disclose a unique integrated combination of a VR devices and mobile processing devices programmed to provide a wide array of neuro-ophthalmologic vestibular assessments, rehabilitation, and training functions.

Currently there is rapid technological growth in the convergence of advanced off-the-shelf (OTS)/VR devices combined with a wide array of OTS of mobile computing devices (MCD) (e.g., smartphones, iPads®, computing tablets, etc.) capable of delivering VR images to the OTS/VR devices. The MCD within the scope of this disclosure contain at least the following: (1) spatial orientation sensors such as such as accelerometers, magnetometers and gyroscopes; (2) programming capabilities within the MCD, and (3) wireless communication of either the MCD's spatial sensor data, and its programming results, with other computing devices. VR capable MCDs are currently manufactured by a growing set of corporations: Apple®, Samsung®, Sony®, Google®, HTC®, LG®, and Motorola®, among others.

The need for more accurate, accessible and cost-effective neuro-ophthalmology, vestibular, ocular and oculomotor assessment devices is well documented by researchers and medical professionals. Current clinical vestibular eye response measuring equipment is highly specialized, bulky, requires a dedicated laboratory and combined is very costly. However, as noted, significant and ongoing advances in both OTS/VR devices and OTS MCDs for those OTS/VR devices provide multiple methods to assess vestibular, oculomotor, and neuro-ophthalmology performance, and the rehabilitation and training based on those performance/assessments/tests.

Previous work in VR using smartphones have been narrow in scope for specific assessments or rehabilitation purposes. However, advances in both OTS VR systems (or augmented reality systems as discussed infra) and MCDs, can now be combined and programmed to provide a novel and wider array of vestibular, oculomotor, and neuro-ophthalmology performance. Thus, the systems and methods of using them disclosed herein keeps pace with the rapid technical advances in OTS VR/MCD; MCDs which have an ongoing increase the spatial sensor accuracy, and both the VR and MCDs with an ongoing decrease in cost per device.

Thus, the need for more accurate, accessible and cost effective neuro-ophthalmology, vestibular, ocular and oculomotor assessment systems is met by the disclosed OTS VR/MCDs in different system configurations: (1) unmodified OTS VR/MCDs, (2) modified OTS/MCD for electrooculogram (EOG) assessments, (3) modified OTS/MCD for precise camera eye tracking, and (4) combinations of them; and the methods of use specific to those systems, providing systems and methods of use for a wide array of neuro-ophthalmologic applications areas such as medical assessments/rehabilitation/biofeedback, competitive athletic training, military applications/training, law enforcement (e.g., user response or eye tracking for alcohol and substance), and job screening or training in occupations requiring fine-tuned balancing and spatial awareness, among other applications of the disclosed systems and methods of use.

### SUMMARY OF THE INVENTION

The following presents a simplified summary of embodiments of systems and methods of their use of the present disclosure in order to provide a basic understanding of such implementations. This summary is not an extensive overview of all contemplated implementations and is intended to neither identify key or critical elements of all implementations nor delineate the scope of any or all claimed inventions. Its sole purpose is to present some concepts of one or more implementations of the present disclosure in a simplified form as a prelude to the more detailed description presented hereafter.

According to teachings of the described embodiments there is provided methods to assess vestibular and ocular performance with unmodified and modified OTS VR/MCD systems.

According to features in the described embodiments there is provided an unmodified OTS VR/MCD system relying on user response to the VR/MCD and/or MCD spatial sensors for the methods of use.

According to yet other described embodiments there is provided modified OTS VR/MCD systems, modified for simultaneous electrooculogram (EOG) integrated into the assessments.

According to further features in the described embodiments there is provided an EOG recording unit and data processor for use in the modified OTS VR/MCD system for EOG assessments.

According to yet other described embodiments are modified OTS VR/MCD headsets using the MCD cameras for tracking eye movements.

According to still further features in the described preferred embodiments is provided modified OTS VR/MCD with embedded micro cameras for tracking eye movements.

According to other features in the described preferred embodiments there is provided various combinations of the above modified and unmodified OTS VR/MCD systems, and the subparts thereof.

According to yet further features in the described preferred embodiments, there are provided various methods of using both the unmodified OTS VR/MCD systems and modified OTS VR/MCD systems to assess visual fields, color blindness, eye movement tracking, convergence, ocular motility, cover-uncover, vestibulo-ocular reflex (VOR), and balance assessments.

Unless otherwise defined here or in the embodiments, all technical and/or scientific terms used herein may have their same meaning as commonly understood by one of ordinary skill in the art to which the term pertains.

Additional advantages and novel features relating to systems and method of use of the present disclosure are set forth in part in the description that follows. The descriptions illustrate embodiments and appended claims taken in conjunction with the accompanying drawings, will reveal more the scope of the disclosure to those skilled in the art upon examination of the following and learning by practice thereof.

### BRIEF DESCRIPTION OF THE DRAWINGS

Other objects and advantages of the invention will become apparent upon reading the following detailed description and upon reference to the drawings in which:

FIG. 1 illustrates embodiments of the instant invention as it might be used in practice.

FIG. 2 is a view of a master menu of representative diagnostic, rehabilitation, training, and other vestibular, oculomotor, neuro-ophthalmology assessments in accordance with methods of using the unmodified and modified VR/MCD system embodiments.

FIG. 3 is a view of a subject wearing an unmodified VR/MCD system capable of wirelessly transmitting yaw, roll, and pitch spatial position data for balance assessments.

FIG. 4 is a view of representative VR images for balance assessments.

FIG. 5 is a high-level flowchart illustrating a method for various balance assessments.

FIG. 6 is a view of representative VR images for testing convergence and divergence assessments.

FIG. 7 is a high-level flowchart illustrating a method for various convergence and divergence assessments.

FIG. 8 is a view of representative VR images for various visual fields assessments.

FIG. 9 is a high-level flowchart illustrating a method for various visual fields assessments.

FIG. 10 is a view of representative VR images for various ocular motility/extra-ocular movement/tracking assessments.

FIG. 11 is a high-level flowchart illustrating a method for various ocular motility/extra-ocular movement/tracking assessments.

FIG. 12 is a high-level flowchart illustrating a method for various vestibular-ocular reflex assessments.

FIG. 13 is a high-level flowchart illustrating a method for various the ocular tracking assessments.

FIG. 14 is a view of representative VR images for various cover uncover assessments.

FIG. 15 is a high-level flowchart illustrating a method for various for various cover uncover assessments.

FIG. 16 is a high-level flowchart illustrating a method for various for color blindness assessments.

FIG. 17 is an inside view of a modified OTS VR/MCD headset with embedded cameras or window openings for forward-facing cameras for eye tracking.

FIG. 18 is a diagrammatic view of an embodiment of an EOG electrodes and photo sensors.

FIG. 19 is a diagrammatic view of an embodiment of an EOG recording unit and data processing device for use with the EOG electrodes and photo sensors of FIG. 17.

FIG. 20 is an inside view of a modified OTS VR/MCD headset with embedded EOG sensors.

FIG. 21 is a high-level flowchart illustrating a method for various EOG assessments.

The embodiments depicted in the figures are only exemplary. One skilled in the art will readily recognize from the following discussion that alternative embodiments of the structures and methods illustrated herein can be employed without departing from the principles described herein.

### DESCRIPTION OF EMBODIMENTS

To the accomplishment of the foregoing and related ends, the invention comprises the features hereinafter fully described and particularly pointed out in the claims. The following description and the annexed drawings set forth in detail certain illustrative embodiments of the invention. These embodiments are indicative, however, of but a few of the various ways in which the principles of the invention may be employed. Other objects, advantages and novel features of the invention will become apparent from the following detailed description of the invention when considered in conjunction with the drawings.

References in this specification to “an embodiment” or “in one embodiment” do not necessarily refer to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance regarding the description of the disclosure.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Finally, it will be appreciated that terms such as “test” and “assessment” used herein are merely for ease of description and refer to a disclosed testing protocol, and as described herein both are within the scope of the present disclosure.

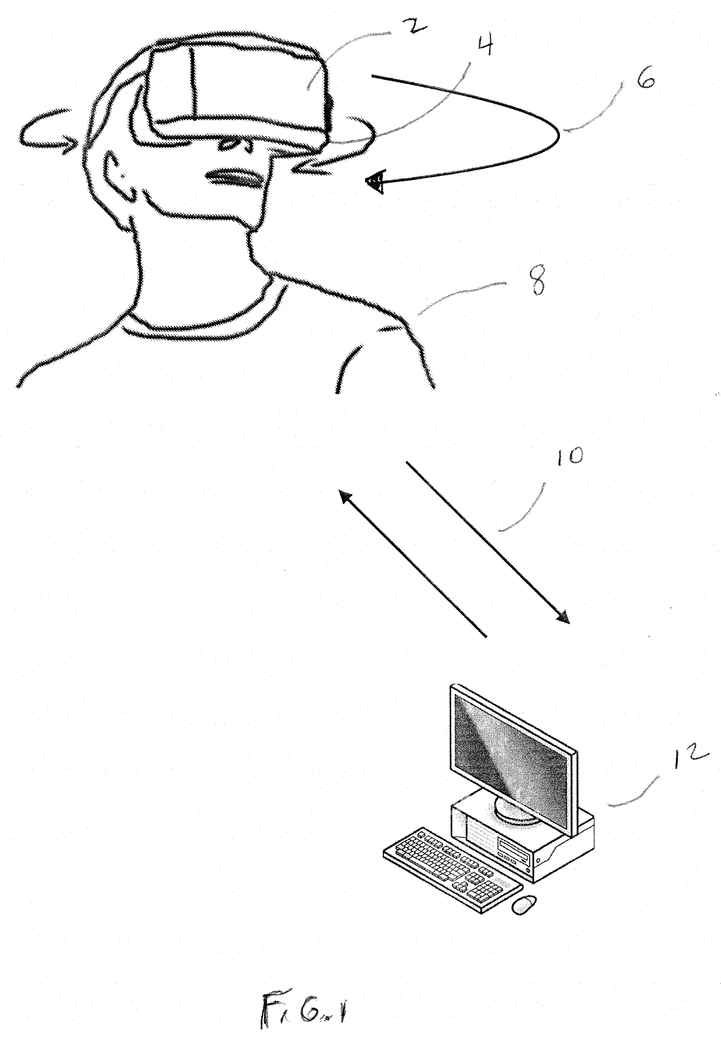

FIG. 1 is an illustration of an exemplary embodiment of both a modified and unmodified OTS VR/MCD system for neuro-ophthalmologic assessments. OTS VR device 4 is combined with the OTS MCD 2, secured to the head of test subject 8, and the combined device is capable of projecting virtual reality images. The OTS VR/MCD systems disclosed herein have two main embodiments: unmodified and modified OTS VR/MCDs. Systems with unmodified OTS VR/MCDs rely on (1) user response to the VR/MCD, and/or (2) MCD spatial sensors for the methods of use. Systems with modified OTS VR/MCD have embodiments with EOG eye electrodes also having embedded photo sensors; embodiments with an EOG recording unit and data processor for use in the modified EOG OTS VR/MCD system; modified OTS VR/MCD headsets using the MCD cameras for tracking eye movements; modified OTS VR/MCD with embedded micro cameras for tracking eye movements; and various system combinations of both the modified and unmodified OTS VR/MCD systems. Additionally, disclosed are various methods of using both the unmodified and modified OTS VR/MCD systems to assess visual fields, color blindness, eye movement tracking, convergence, ocular motility, cover-uncover testing, VOR, and balance assessments, among other methods of use (e.g., related training, biofeedback, and other applications.)

In all the embodiments, and combinations thereof, neuro-ophthalmologic assessments are programmed either within the MCD 2 and/or transmitted wirelessly 10 to an OTS computing device 12 for further test interaction during an assessment, further processing, storage, or off-line processing.

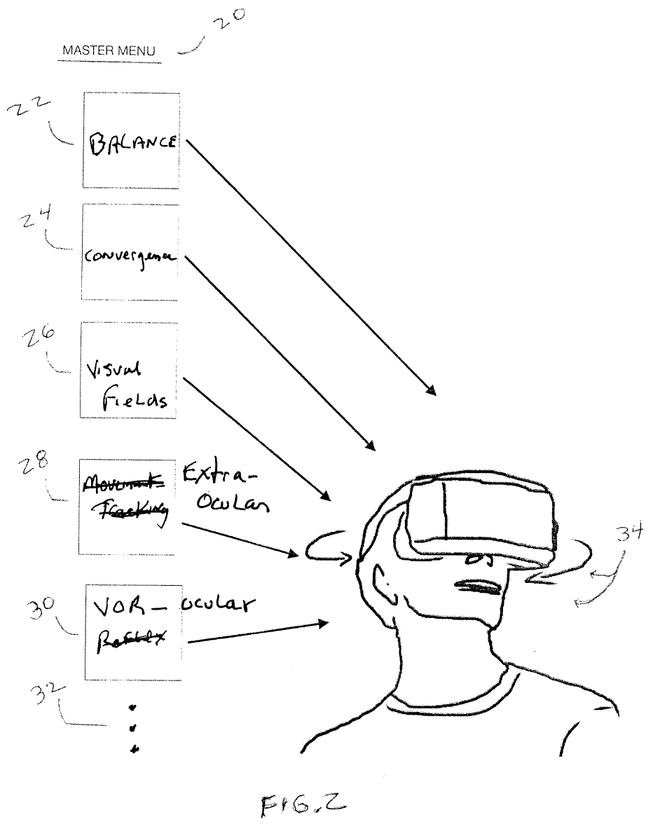

FIG. 2 is a view of a sample master menu of possible diagnostic, rehabilitation, training, and other vestibular, oculomotor, neuro-ophthalmology assessments in accordance with principles of the programable OTS VR/MCD, modified or unmodified, of the present disclosure, systems. The master menu 20 can comprise of balance 22, convergence 24, visual fields 26, extra-ocular 28, VOR 30 assessments, among a wide variety of other possible vestibular, oculomotor, neuro-ophthalmology assessments 32, as disclosed below.

Disclosed embodiments include both OTS VR and augmented reality (AR) devices. VR generates an immersive, artificial, computer-generated simulation of a real life, and AR layers computer-generated reality-enhancements atop existing reality so the user interacts with both. Both therefore have applicability in the above listed OTS systems for vestibular, oculomotor, neuro-ophthalmology assessments, rehabilitation, and training applications. As used herein “virtual reality” (VR) covers both virtual reality and augmented reality.

As noted, VR headsets combined with MCDs are both decreasing in cost and with increased spatial sensor capabilities (see, e.g., Ma, Z., Qiao, Y., Lee, B., Fallon, E., Experimental Evaluation of Mobile Phone Sensors, Conference paper, 24th IET Irish Signals and Systems Conference (IS SC 2013)). These MCDs with a growing increase sensor accuracy of spatial position data thus provide increased programmability to track, for example, postural changes during balancing. Most of the global corporations manufacturing the current and next generation MCDs are increasing such sensor precisions, and providing greater resolution in their MCD dual cameras (e.g., forward and rear-facing cameras). All these current and projected OTS VR and MCD capabilities (either separately or in a VR/MCD combination) are within the scope of the disclosed embodiments.

In both the unmodified OTS VR/MCD embodiments the VR/MCD is secured over the user's eyes, presents VR images to the user within the VR/MCD creating the perception of a 3-dimensional (3D) stereoscopic environment. The VR images presented are either from stored content within the MCD, from the wirelessly connected computing device, or from high bandwidth streaming over a network. Disclosed embodiments utilize that VR imagery for assessment of neurology neuro-ophthalmologic functions.

The combined VR/MCD in the disclosed embodiments include three main ways of VR/MCD interacting: first, test subject 8 physical interaction with virtual world of the device; second, the virtual interactions between the test subject 8 and algorithms running within the VR/MCD, and third, test subject 8 interacting with someone monitoring the assessment at wirelessly connected computing device 12, the monitoring agent communicating with the test subject either through computing device 12 or verbally, and can iterate assessments based on that interaction.

Additionally, wirelessly connecting computing device 12 interfaces with either the combined OTS VR/MCD system, or with the MCD. This interaction is provided via readily available MCD sensor data apps which provide its ‘real-time’ (or near ‘real-time’) spacial position, spatiotemporal, and spatiotemporal dynamic data to computing device 12. Most OTS MCD have a native code interface, e.g., Java®, and easily interconnected with a programing language on computing device 12, e.g., via Python®, C/C++®, among others, and either the processing within the OTS VR/MCD system or through its wireless interface with computing device 12 can set sampling rates for each assessment, and the number of iterations per assessment.

Overall, both the OTS VR and OTS MCD devices have readily available input and output (I/O) interfaces that combined provide the flexibility of the unmodified OTS VR/MCD embodiments disclosed herein. That is, the MCD and VR systems within the scope on the disclosed embodiments readily connectable own I/O interfaces for the above noted methods of using all system embodiments. Each system embodiment, and the combinations thereof can also send raw sensor data, processed preprocessed and sent wirelessly to computing device 12 (e.g., desktop computer) or any available wireless computing device, for analysis, further analysis based on the processing done in the VR/MCD, and storage for yet further ‘off-line’ analysis.

For purposes providing the extemporary embodiments disclosed herein, the algorithms will work with embedded accelerometer data, which is a useful for monitoring sensor date for monitoring head movements of test subject 8 (but other sensors like the gyroscope or rotation vector sensors, or other spatial, or spatial-temporal sensors, among others, are also applicable).

Unmodified OTS VR/MCD Assessments

As noted, unmodified OTS VR/MCD embodiments are capable of programming a wide variety of vestibular, oculomotor, neuro-ophthalmology assessments, e.g., among others, for balance, convergence/divergence, visual fields, extra-ocular/extra-ocular movement, vestibular-ocular reflex, ocular tracking, cover-uncover, and color blindness assessments (testing), rehabilitation (including biofeedback), and training. The methods of use of in unmodified OTS VR/MCD embodiments are through user responses, e.g., test subject 8, to the unmodified OTS VR/MCD embodiments, and/or with OTS VR/MCD sensor data. The following methods of use of the unmodified OTS VR/MCD embodiments provide one of ordinary skill a sense of the range of that wide range of programmability of the unmodified OTS VR/MCD embodiments for those vestibular, oculomotor, neuro-ophthalmology functions, and a wide range of their applications. Further, the unmodified OTS VR/MCD is capable of a wide range of vestibular, oculomotor, neuro-ophthalmology assessments, rehabilitation, and training applications at a significantly low cost per device versus current either the narrowly focused VR assessment systems or bulky high cost, current state-of-the-art, vestibular, oculomotor, neuro-ophthalmology systems.

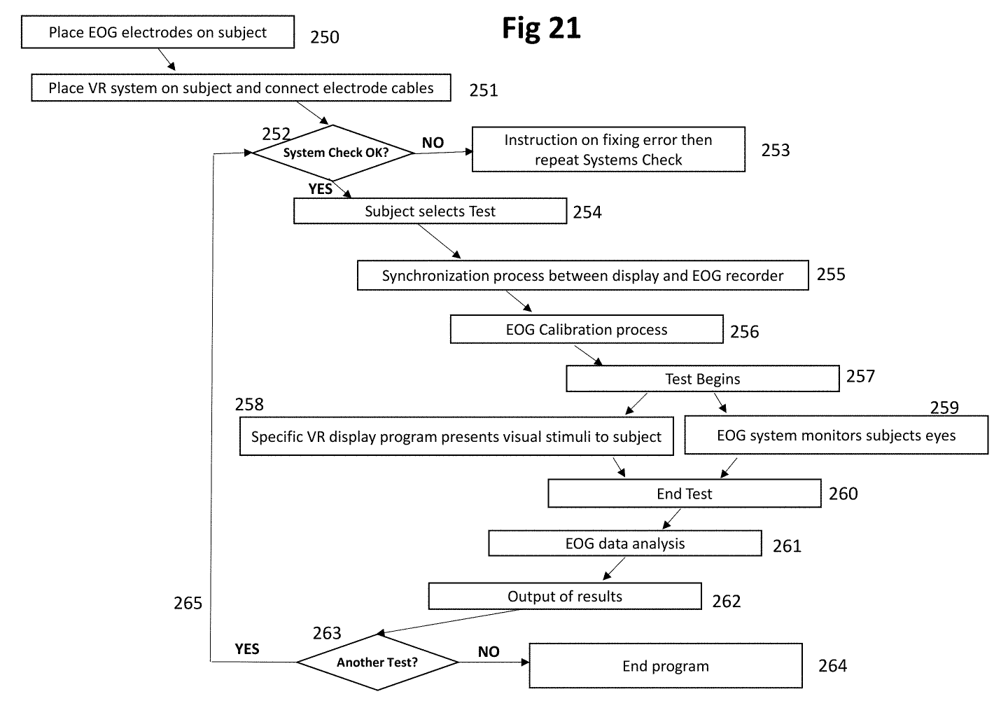

Also, methods of use for the unmodified OTS VR/MCD system embodiments also apply to the methods of use for the modified OTS VR/MCD system embodiments. The listed methods of use for the modified OTS VR/MCD system embodiments will adjust appropriate I/O variables, for example the EOG electrode and photo sensor data, as disclosed below. But, similar spatiotemporal coordination between the VR imaging and the sensor data for the balance tests apply to both unmodified and modified OTS VR/MCD system embodiments with adjusting for the different VR and/or MCD sensor data for spatiotemporal analysis per each method of use. For example, in eye tracking embodiments, test subject 8, eye movements are assessed, and thus the tests do not primarily rely on test subject 8 responses for those methods of use. Similarly, refinements in the methods of use for EOG embodiments are disclosed in FIG. 21.

Balance as Sesements and Training

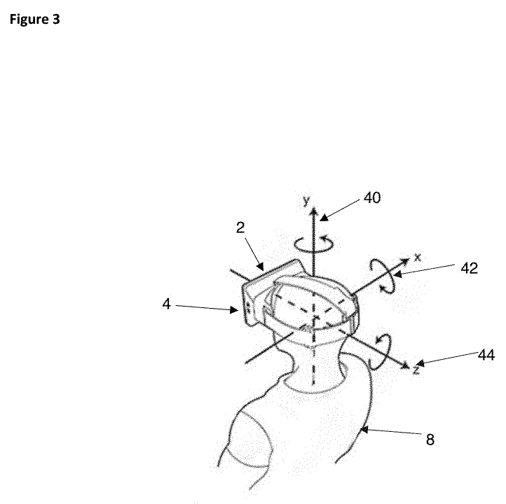

FIGS. 3, 4, and 5 depict certain aspects and features of the unmodified OTS VR/MCD system embodiments and balance embodiments of methods of use of those systems. Balance testing assesses a person's ability to maintain a stable posture in an upright position. Balance is controlled by complex processing of the nervous system, relying on three sources of primary sources of information (1) vestibular input from the inner ear, (3) proprioceptive sense from the tendons, ligaments, and muscles of the body, and (3) vision. Thus, balance responses involve and provide assessments of all those, and related physiological systems.

FIG. 3 illustrates an representative configuration of unmodified OTS VR/MCD system embodiments to measure balance. The unmodified OTS VR/MCD 2 and 4 present VR images to the test subject 8 and sensor data within unmodified OTS VR/MCD 2 and 4 transmit (or preprocess and then store or transmit) the head/body movements of the test subject 8. These movements, combined with the VR imaging, provide the balance algorithm with spatiotemporal data for the balance assessments. That sensor data, in different embodiments, (other embodiments use other spatial MCD sensors, for example, a gyroscope or digital compass) provides spatiotemporal x 42, y 40, and z 44 locations of test subject 8 head movements during the test, that is yaw 40, pitch 44, and roll 42 data from the MCD accelerometer, for processing, as noted, within the combined VR/MCD 6, or in the wirelessly connected 10 computing device 12. Additionally, a clinician or user at computing device 12 may interact with the ongoing testing, execute different tests, with different VR simulation/assessment imaging.

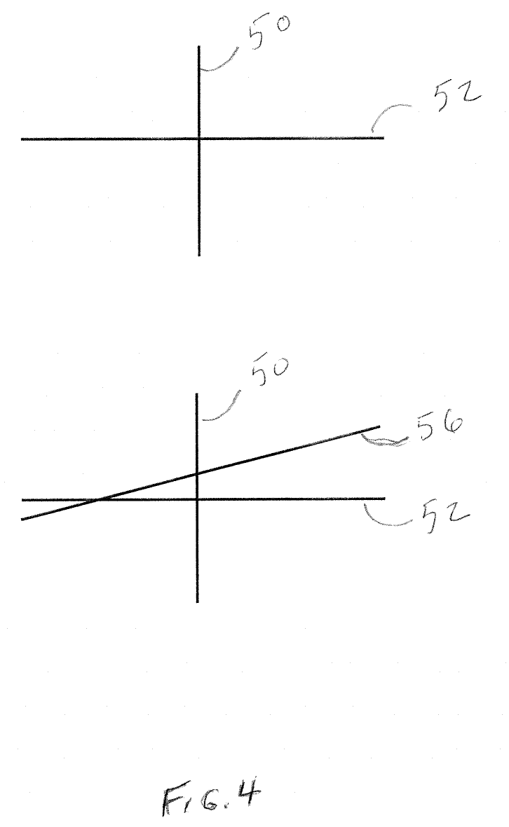

In balance assessments, for the purposes of disclosing the specifics of this embodiment, those assessments use accelerometer data coordinated with test subject 8 in various postural positions, coordinated with different VR imaging, including static true-horizon environments, moving environments (for example rocking or spinning environments) and dark environments. For example, test subject 8 can either be assessed with traditional balance assessment (e.g., a Rhomberg Test), or, as shown in FIG. 4, one or more VR images displaying the sway of a VR horizontal horizon from 52 to 56, providing a static true-horizon scene 50 and 52, and a moving horizontal horizon 56, swaying about a fixed VR axis 50.

Specifically, for this representative embodiment head/body movement is collected from the MCD accelerometers to assess the amount of postural sway or movement assessment during the test. Various balance test protocols are within the scope of this disclosure, e.g., Static Postural Sway, Tandem Stance, Tandem Gait, Single Leg Stance, Dynamic Squat, Single Leg Squat, Step up Test, Up and Go Test, Jump Stability, or as noted, the Rhomberg Test. For either protocol, postural sway and stability with unmodified OTS VR/MCD embodiments provide accurate response assessments.

Those of skill in the art will appreciate multiple exemplary ways of processing sensor movement data, as taught in U.S. Pat. No. 7,292,151 which is incorporated herein by reference. Additionally, see Eager, D., Pendrill, A. M., and Reistad, N., Beyond velocity and acceleration: jerk, snap and higher derivatives, European Journal of Physics, Volume 37, Number 6. In one embodiment, readings from the MCD are processed for displacement, x, the velocity of that displacement, v, where v=dx/dt, its acceleration, a, where a=dv/dt, and the rate of change of the acceleration, commonly called jerk, is da/dt (i.e., the third derivative of the head displacement).

As noted, in other embodiments, the testing protocol assesses sampled ‘real-time’ accelerometer data, records that data, and analyzes it to provide that assessment of postural sway and stability, and provides different balance scores, like the total movement during the test, and how skewed that total movement is, the asymmetrical distribution of the acceleration data (i.e., another way to assess ‘jerkiness’ of the movement). Whatever math is used, among the variety cited to process postural movement for different balance protocols, the given numerical/quantitative results of each are obtained for either (1) a clinician/user at computing device 12, or (2) for biofeedback within the VR device to the test subject 8.

In one embodiment, embedded in the OTS MCD provides gravitational force changes in the spatial 3D, 3 axis, 40, 42, 44 dimensions (FIG. 3) providing the test subjects head movement and position changes. In this embodiment, accelerometer data is collected during each testing condition. The data is transformed through computational algorithm locally on the device or in an internet connected computer or server to yield a number or set of numbers which reflect the motion or changes in posture of the user. This data is then stored locally on the device or in the server or transferred from device to server. The user, or test subject 8, is instructed to maintain a specific posture and built-in accelerometers in the VR/MCD system measured g-force acceleration in the three dimensions.

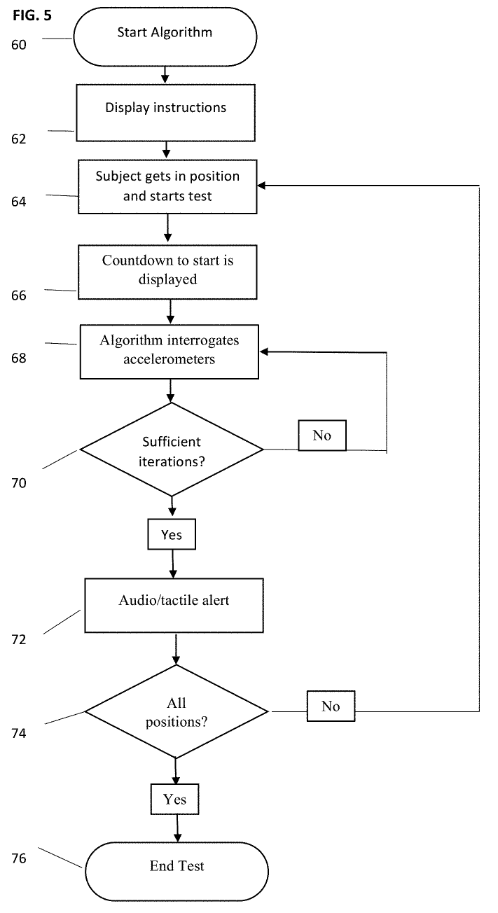

Thus, in general, the purpose of one embodiment of the balance assessment is to identify (1) absolute amount of movement, and to (2) compare that movement with a statistical model/estimated norm, an average, of balance. FIG. 5 discloses is a high-level flowchart illustrating a method for this embodiment, a test subject 8 initiated assessment within the VR environment. Instructions to the subject are displayed in VR scenes within the MCD, and may include instructions description of tests, stances and conditions (static environment, moving environment, dark environment, etc.) of that balance assessment. Once the balance assessment begins 60, test subject is able to select assessment items on a different VR menus, and initiate the test 62, either by head movements, i.e., holding a VR cursor for a given length of time on a menu item, or with the test subject 8 manually initiating an assessment by a tap 64 of the control pad or remote button press. Then, in one embodiment, test subject 8 sees, for example a countdown to the start of the test, allowing them to prepare and position themselves for the assessment during the countdown displayed on the VR screen. The balance algorithm then interrogates the MCD accelerometers 68 during the test, each test having within a given test period, and the accelerometer data sampled at a given sample rate during that test time. Thus, the loop from 70 to 64 iterates during that test period, collecting the sampled accelerometer data.

When a particular test period and balance assessment is finished, 70, the test subject 8 is notified, either in the stand alone mode via the VR screen or by a clinician at the monitoring computing device 12. This process of sampling the accelerometer is repeated for each balance stance/condition, loop 70 to 64. Results are calculated, stored locally in smartphone memory, and/or transmitted to internet storage 72, or results are displayed locally on the MCD display and/or remote computing device 12.

In one embodiment, the distance between each sampled 3D displacement is calculated using Pythagorean theorem distance difference between each x, y, z accelerometer reading (e.g., a moving 3D set of triangles in space with the difference, the displacement, between each being the hypotenuse of each). Those difference or displacement samples are stored during each test providing an accumulated list of the postural displacements during the test. Each is displacement variable adjusted to a total movement distance during the test by calculated as the absolute value of each, providing the total sway or displacement during the test (i.e., an arithmetic mean, the absolute value of the displacements divided by the total number of displacement sampled) providing the total distance moved per test (i.e., how steady the test subject was during the test).

Additionally, a root mean square of that total displacement provides the amount of jerkiness of the postural motion during the balance assessment, (the root mean square of arithmetic mean of the sampled data showing how skewed or asymmetrically the movement is distributed versus a statistical mean or norm of movement per the particular balance test).

And, as noted, in other embodiments, similar algorithms of postural sway and motion provide biofeedback for rehabilitation or balance training, or for a learning paradigm, making the test subject 8 aware of those test scores, which he or she may not normally be conscious so that they can influence or improve that function.

In the context of the disclosed embodiments, biofeedback refers to the method of making a user aware of information about body function regarding balance, convergence, visual fields deficits, extra-ocular movement, tracking and targeting, and vestibulo-ocular reflex function in a training paradigm for the purpose of improving those functions.

As noted, these assessment use the same OTS VR/MCD embodiments disclosed above, and similar balance data collection and processing. But in the training or biofeedback embodiments, the collected data is used to provide information and visual or auditory feedback as a method of biofeedback training for improving postural balance.

Convergence/Divergence as Sesements and Training

Convergence divergence testing is well known neuro-ophthalmologic diagnosis of a variety of neuro-ophthalmologic vestibular disorders. Overall, visual fusion as used herein means the combining of images from the two eyes to form the perception of a single object.

In one embodiment, a convergence/divergence assessment the movement of the two eyes to come together or move away from each other in response to VR imaging to maintain single binocular vision of that object as it moves closer (convergence) or farther away (divergence). As disclosed herein, this object convergence and divergence (for both distant and near VR objects) is via the VR imaging, and the spatiotemporal coordination between the VR imaging and the sensor data is assessed for those diagnostics.

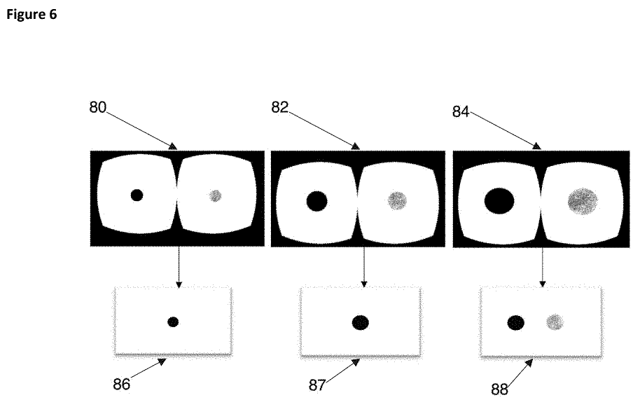

FIG. 6 discloses the test subjects 8 VR view, in a representative embodiment, where the top row of images 80, 81 and 82 demonstrates how a target object may be presented to each eye individually with the object moving progressively closer from 80 to 81 to 82, and the perception of those VR images 84, 86, and 88 remain fused as one object until the object gets too close and the subject 8 can no longer maintain fusion and indicates a perception of the double images 88. Testing consists of determining at which point test subjects 8 can no longer maintain convergence as the virtual object is perceptually close.

FIG. 6 provides a representative example object on the screen to give the perception of the single fused object moving closer and farther away. When the object appears too close to test subject 8, test subject 8 notes the break in convergence when the two images are no longer fused. Thus, the method of use in this embodiment is by user response (instead of more costly eye tracking embodiments). Thus, test subjects 8 provides a verbal, physical signal, response on a keypad, or, among other ways, to a response pad on the unmodified OTS VR/MCD system, signifying when convergence is or is not maintained, depending on the convergence/divergence testing protocol.

Likewise in convergence training, using unmodified OTS VR/MCD embodiments, the collected data, spatiotemporal coordination between the VR imaging and user provided response is used to provide information and visual or auditory feedback as a method of biofeedback for improving convergence performance in an exercise or training format.

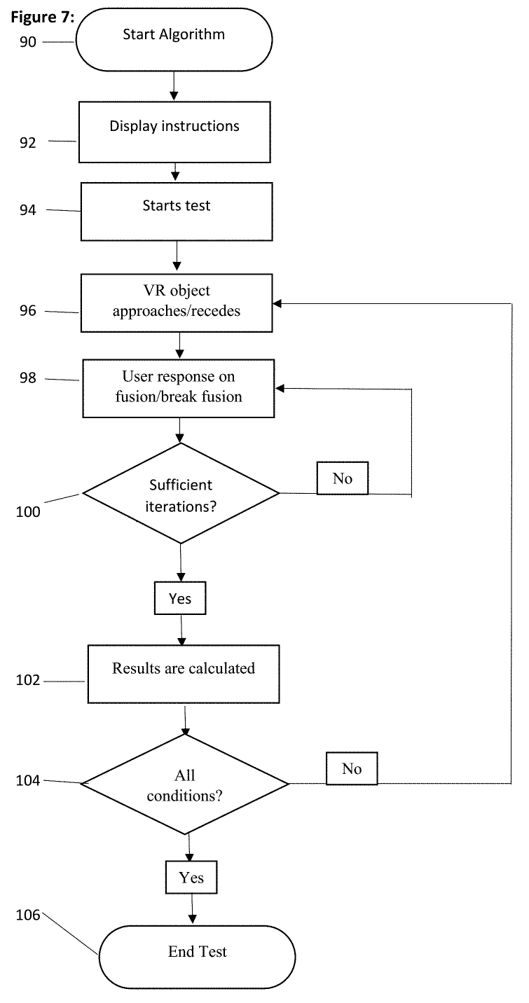

FIG. 7 discloses an embodiment of a high-level flowchart illustrating a method for various convergence and divergence assessments. The assessment protocol begins 90, instructions to test subject 8 are displayed on the VR/MCD, and may include description of tests, how the subject is to respond (e.g., by touch pad or remote control). Then the test is initiated by test subject 8, e.g., buy a tap of the control pad or remote button press. Thereafter, test subject 8 sees VR images 96 creating the perception that an object or objects are approaching or moving away from the subject in via VR imagining. Thus, the VR object is displayed as approaching 98 and test subject 8 indicates by touch pad or remote button press when the object is perceived as double. The algorithm then calculates the average virtual distance of the object to the eyes at the time of the subject's response 100. Results are calculated 102, stored locally in MCD memory, and/or transmitted to for off-line storage and/or further processing. The convergence testing of the VR images is then repeated, 102 to 96, as needed, and the results are averaged and stored.

Further, in one embodiment, a VR image, similar to those in FIG. 6, are then displayed as moving away from test subject 8, who likewise as in the convergence test indicates by touch pad or remote button press when the object is perceived as fusing from a double to a single image. Thereafter, algorithm 100 calculates the virtual average distance of the object to the eyes at the time of test subject 8 response. Those results are stored locally in the MCD memory, and/or transmitted to for storage, and and/or further processing 108. The results of the each test are also averaged and stored in the MCD memory, and/or transmitted to for storage, and and/or further processing 108. Finally, those results may then be displayed within the VR environment 110 for, among other reasons, for biofeedback to test subject 8.

Visual Fields Testing

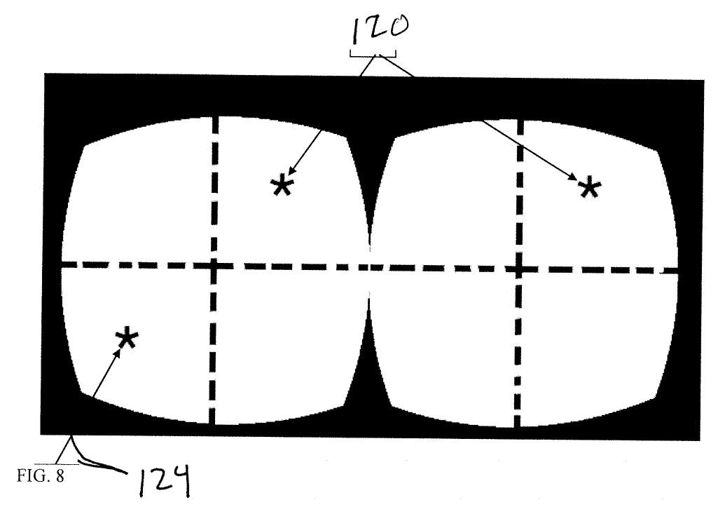

As is well known in the art, the perceptual field of vision may be interrupted at any point in the path between the retina and the primary visual centers of the brain and provides additional wide range of diagnostic assessments. FIG. 8 illustrates the presentation of visual stimuli for visual fields testing either by presentation of VR stimuli in both eyes simultaneously 120 or in on eye only 124. Thus, in visual fields testing the VR images create the perception of objects that appear in different regions of the visual fields, either in one eye alone or in both eyes.

In one embodiment of the unmodified OTS VR/MCD system, measurement of the integrity of the visual fields through test subject 8 response. As in the above, test subject 8 provides a verbal or physical signal or response on a keypad, response pad or controller as to whether he or she sees the VR object or not. And based on that response a reaction time is measured to provide a further assessment of brain function.

Likewise in visual fields training, using the same OTS VR/MCD embodiment as above. The visual field data and processing provides visual or auditory feedback as a method of biofeedback for improving visual field performance in an exercise or training format.

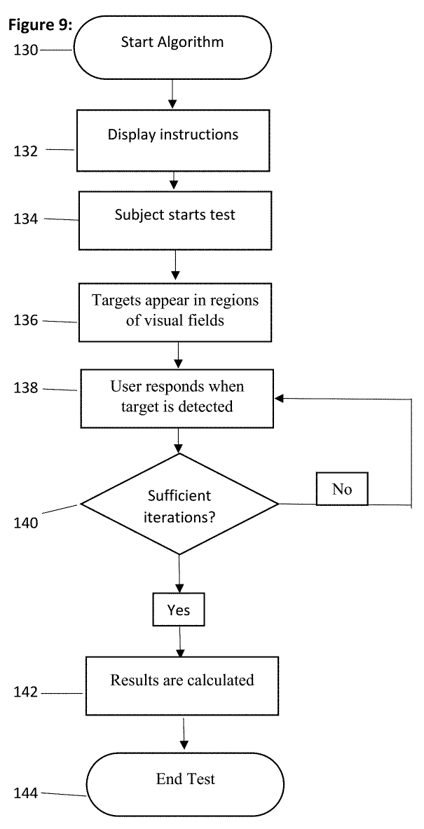

FIG. 9 is a high-level flowchart illustrating a method for various visual fields assessments. The visual fields assessment begins 130, and again, instructions may be displayed to test subject 8 on the VR/MCD device, may include a description of tests, and how test subject 8 is to respond (e.g., by touch pad on the VR/MCD device, or remote control). In one embodiment, the test is initiated by touch pad on the VR/MCD device 134.

As illustrated in FIG. 8, representative VR images create the perception of objects that appear in different regions of the visual fields 136, either in one eye alone or in both eyes, while test subject 8 is presented a center visual fixation point. Thereafter, in one embodiment, test subject 8, responds, as noted, by user response to the VR/MCD system, the moment the VR object/or objects appear 138. The algorithm then calculates hits and misses recorded versus the location of VR target object, measuring the reaction time of correct hits 140. Results are then calculated, stored locally in stored locally in the MCD memory, and/or transmitted to for storage, and and/or further processing 142. And the visual fields test is repeated, as needed, to cover all regions of the visual fields. The results of the number of iterations of visual fields tests run 142 to 136 are averaged and stored in memory, and results of the locations of hits and misses and the average reaction times are displayed locally 144 on the VR/MCD display and/or transmitted wirelessly to computing device 12 for off-line storage and/or further processing.

Ocular Motility/Extra-Ocular Movement Testing

As is well known in the art, ocular motility testing assesses the quality of eye movements, and how the two eyes move together as they follow a target, and those assessments allow for the diagnosis of, among other things, strabismus, extra-ocular muscle dysfunction, or of the cranial nerves which innervate the extra-ocular muscles.

In the unmodified OTS VR/MCD embodiments, measurement of ocular motility/extra-ocular motility through user response. VR images are presented in one eye alone, in both eyes, or differently in one eye than the other to further create the perception of objects or images in the visual fields.

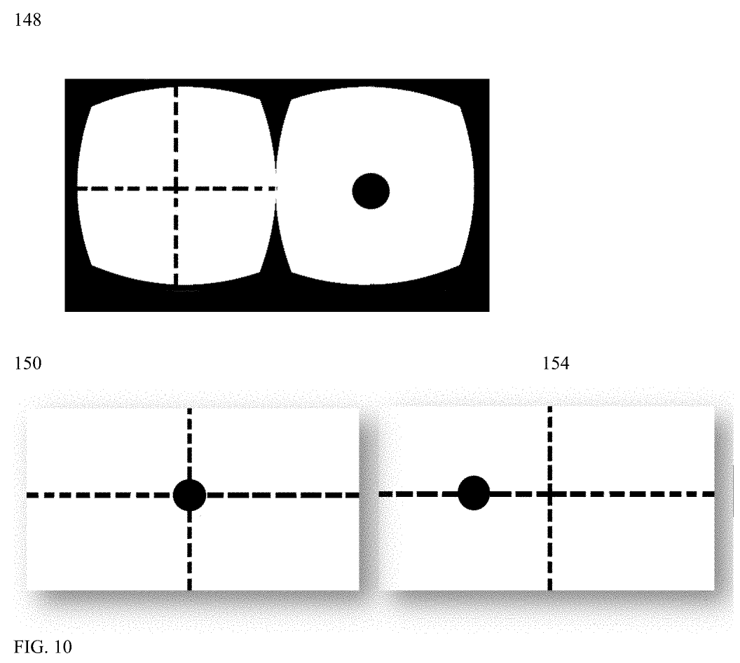

FIG. 10 is a view of representative VR images for various ocular motility/extra-ocular movement/tracking assessments. In one embodiment, a crosshair image is presented to the right eye 148 (left side of FIG. 148). Test subject 8 is instructed to fix gaze on the center of the crosshairs, and an image of an object is presented to the left eye 148 (right side of the FIG. 148), centered on the crosshairs. Test subject 8 then indicates whether he or she perceives it on the center of the crosshairs, or in one of the quadrants created by the crosshairs. The center point of the crosshairs then moves to different positions in the visual fields and test subject 8 indicates whether the object remains on the crosshair center point or moves to one of the quadrants. Test subject 8, as noted above, provides responses noted, for example, via user response to the VR/MCD system, as instructed, whether the object appears on the crosshair center point or in which quadrant it appears.

Software algorithms then analyze those responses to provide an assessment of whether or not the eyes are aligned properly. For example, if the eyes are not aligned properly the pattern is consistent with dysfunction of the cranial nerves that operate the ocular motility/extra-ocular movement. Or, if responses are not consistent with a cranial nerve dysfunction, other dysfunctions of ocular motility/extra-ocular movement are considered with the user responses (i.e. Strabismus or Skew Deviation).

Likewise in ocular motility/extra-ocular movement training, using embodiments as disclosed above, the collected data is used to provide information and visual or auditory feedback as a method of biofeedback for improving ocular motility/extra-ocular movement performance in an exercise or training format.

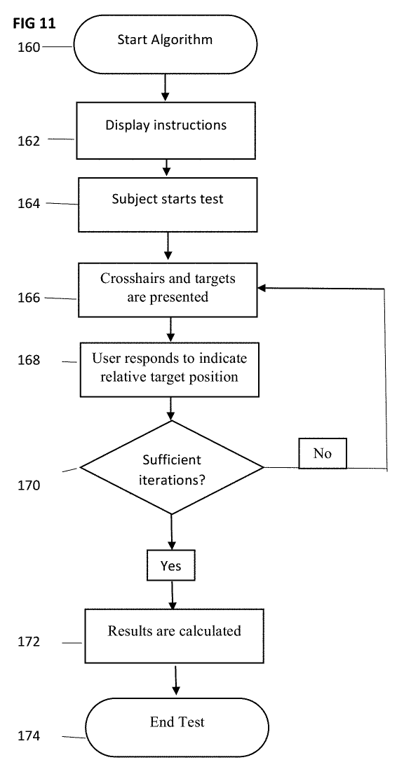

Further, FIG. 11 discloses a high-level flowchart illustrating a method of use for various ocular motility/extra-ocular movement/tracking assessments. The ocular motility/extra-ocular movement/tracking assessments begins 160, and instructions to the subject are displayed in the OTS VR/MCD system. These instructions include description of tests and how the subject is to respond 162. In one embodiment, the test is initiated by test subject 8 to the OTS VR/MCD system 164. VR images are then presented in one eye alone, in both eyes, or differently in one eye than the other to further create the perception of objects or images in the visual fields. In one embodiment, a crosshair-image is presented to the subject's right eye, and test subject 8 is instructed to gaze at the center of the crosshair 166.

Target objects are presented in the location of the center of the crosshair in the visual field of the right eye. Test subject 8 responds to indicate whether the object appears in the center of the crosshairs, above or below the horizontal, to the right or to the left of the vertical, or in one of the four quadrants created by the crosshairs 168, and ocular motility/extra-ocular movement/tracking is repeated 168 to 166 a desired number of iterations, with the center of the crosshairs moving to different orthogonal points of the visual fields of the right eye. Results are calculated, stored locally in OTS VR/MCD system memory, and/or transmitted to wirelessly for storage 170, and the algorithm provides an analysis of the oculomotor dysfunction based on the responses of the subject 172.

The different ocular motility extra-ocular movement tracking then provides the following results: if the subject perceives the object to the right of the vertical crosshair, the algorithm reports exotropia or exophoria; if this worsens as the crosshairs move to the right then a Medial Rectus muscle weakness and/or Third Nerve palsy is suggested; if the subject perceives the object to the left of the vertical crosshair, the algorithm reports an esotropia, if it worsens as the crosshairs move to the left then a Lateral Rectus muscle weakness and/or Sixth Cranial Nerve Palsy is suggested; if the object appears above the horizontal crosshair, the algorithm reports a hyper-deviation, if it worsens as the crosshairs move to the right then a Superior Oblique muscle weakness and/or Fourth Cranial Nerve Weakness is suggested; if the object appears below the horizontal crosshair, the algorithm reports a hypo-deviation, if this worsens as the crosshairs move to the left then a Inferior Rectus and/or Third Nerve palsy is suggested; if the object is superimposed on the crosshair center, the algorithm reports no vertical or horizontal deviations present.

In one embodiment, the test is repeated with the crosshairs presented to the left eye and the target object presented to the right eye 174 to 166, and the flowchart algorithm provides an analysis of the oculomotor dysfunction based on the responses of test subject 8. With the same response assessments noted above based on the testing of the left eye.

Finally, results are displayed locally on the VR/MCD display 176 and/or transmitted wirelessly to computing device 12 for off-line storage and/or further processing.

Vestibular-Ocular Reflex Testing

VOR testing assesses eye movements that function to stabilize gaze, by eye movement counter to head movement. As is known in the art, VOR tests complex coordinating eye reflex movements in response to movements of the head, gravitational and acceleration forces on the vestibular apparatus of in the inner ear, mediated through brain structures such as the brainstem, cerebellum and cerebrum and thus can diagnosis a wide variety of VOR related maladies.

In one embodiment of the VOR testing, using the unmodified OTS VR/MCD display, a VR image of an object(s) is presented as a fixation point. Data is collected from the accelerometers embedded in the device regarding gravitational and acceleration force changes in 3D axises related to the movement or change in test subject 8 head positions. In the autorotation test, test subject 8 is instructed to turn his head side to side or move the head up and down while keeping his eyes on the fixation point or test subject 8 is placed in different positions such as lying on one side, or tilting his or her head in one direction or another.

In one embodiment, assessment of vestibular-ocular reflex testing is through user response, using responses noted above. Test subject 8 provides those responses indicating when he or she perceives movement, spinning, or vertigo in various conditions: (1) changes in body positions: upright, sitting, lying in supine or non-supine positions; (2) changes in head position: head centered, turned to the left or the right, looking upwards, or downwards in a static or oscillating pattern (may be combined with (1) above; and (3) exposure of changes in temperature to the outer ear canal (i.e., warm or cold air or water placed in the canal).

In one embodiment, during autorotation the object or fixation point may change briefly in appearance. Test subject 8 is instructed to respond each time the object changes. Reaction time is recorded as an independent measure of nervous system function. Dysfunction of the vestibular-ocular reflex will cause interruption of gaze on the fixation point or object and the user will not perceive the change in appearance resulting in an error of omission of the response.

Embodiments for vestibular-ocular reflex training, utilize disclosed system and method embodiments above, and provide visual or auditory feedback as a method of improving vestibular-ocular reflex response and performance in an exercise or training format.

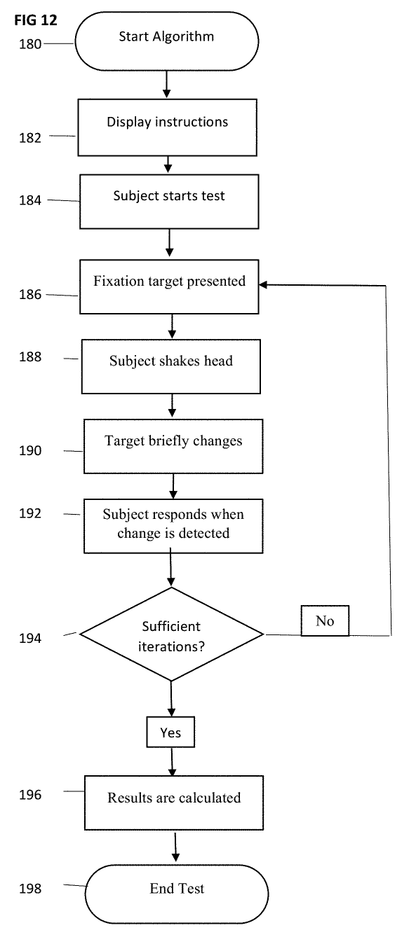

FIG. 12 is a high-level flowchart illustrating a method of use of the OTS VR/MCD system for various vestibular-ocular reflex assessments. The algorithm begins 180, instructions to test subject 8 are displayed on the VR/MCD, and may include a description of tests, how test subject 8 is to respond (see above). And in one embodiment test subject 8 initiates the test by a tap of the control pad or remote button press 184. Then, a VR image of a fixation target is presented in the virtual environment 186. Test subject 8 is instructed to shake their head right and left in a “no-no” pattern and maintain gaze fixation on the target object 188, and test subject 8 times the speed of the movements to a metronome style audio output 190. While test subject 8 maintains gaze on the target object it changes in appearance briefly (for example a color change, or the appearance of a letter or number as a target) 192. Testing subject 8 by user response indicates each time the object changes in appearance 194. And from these responses, correct and incorrect responses and reaction times are recorded and stored in the VR/MCD device 198, and/or transmitted wirelessly to computing device 12 for off-line storage and/or further processing.

The test is repeated with the subject moving the head in an up and down direction in a “yes-yes” pattern with gaze fixation on the virtual target object, and with the head placed in a position such as tilted to the left or to the right or lying with the head turned to one side or another or when cold or warm water/air is applied to the ear canal 196 to 186, and again results are displayed locally on the VR/MCD device display 198 and/or computing device 12.

Ocular Tracking Test

As noted, unlike current bulky and expensive equipment to track eye movement, disclosed system embodiments of the unmodified OTS VR/MCD system track eye movement with a significantly reduced price per device and thus make it applicable for a wider array of uses, both in and out of a clinical setting. Overall, as is well known in the art, eye tracking is the process of measuring either the point of gaze (where one is looking) or the motion of an eye relative to the head. An eye tracker device measures eye positions and eye movement during those eye tracking tests.

In one embodiment, test subject 8 is presented with the VR image of an object moving across and around the virtual environment. The VR image may briefly change in appearance in some substantial form. If ocular tracking is impaired, the subject may not perceive the brief change in appearance of the target, and in the unmodified OTS VR/MCD system the measurement of ocular tracking through user response (which, among other aspects provides the significant cost reduction of the device). Thus, in some embodiments of the unmodified OTS VR/MCD system test subject 8 provides that response, among other ways, with a verbal or physical signal or response on a keypad or controller as to when they see the VR test object change in appearance, and algorithms record the accuracy of the responses and reaction time.

Similarly, in biofeedback or training embodiments, ocular tracking, as described above, provide information and visual or auditory feedback to test subject 8 for improving ocular tracking response performance improvements, in an exercise or training formats.

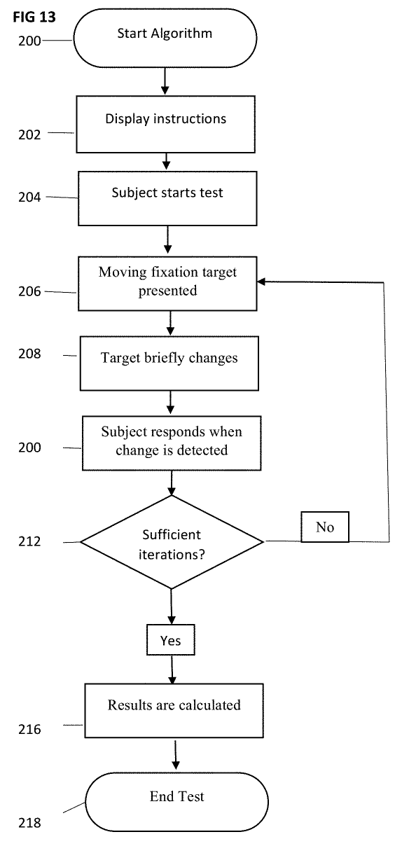

FIG. 13 shows a high-level flowchart illustrating a method for various the ocular tracking assessments. The ocular tracking assessment begins 200, and instructions to test subject 8 are displayed on the unmodified OTS VR/MCD device. These instructions include description of tests, how test subject 8 is to respond (by touch pad or remote control) 202. In the user response mode test subject 8 initiates the test 204. Thereafter, test subject 8 is presented with the VR image of an object moving across and around the virtual environment 206. This image may briefly change in appearance in some substantial form. If ocular tracking is impaired, the subject may not perceive the brief change in appearance of the target 208. And, as noted, in user response mode, test subject 8 indicates when a change in the target object is detected 210. Correct and incorrect responses and reaction times are recorded and stored in stored in the VR/MCD device 212, and/or transmitted wirelessly to computing device 12 for off-line storage and/or further processing.

Additionally, ocular tracking is repeated with varying speed of the movement of the virtual object 212 to 206. And again results can be displayed locally on the unmodified OTS VR/MCD display 214 transmitted wirelessly to computing device 12 for off-line storage and/or further processing.

Cover Uncover Testing

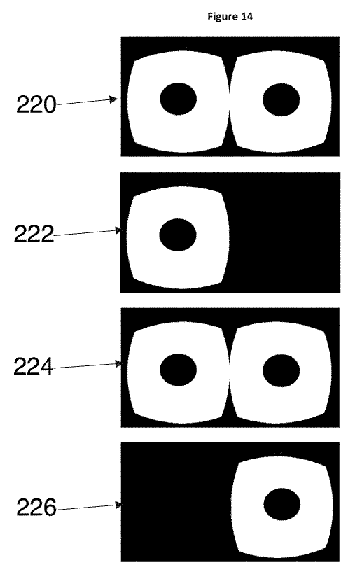

In cover/uncover testing, images presented in the near vision VR environment where the target image is presented in the same location for both eyes. FIG. 14 discloses possible embodiments of VR images for various cover uncover assessments. In one embodiment, first VR images are presented to both eyes with the target in the same place for both eyes 220. Then the image in one eye is blacked out 222. The images are presented in both eyes again 224 and then the image is blacked out in the other eye 226. Thus, images presented one of the eye is replaced with a completely dark screen, or the target image may simply be removed from presentation in one eye. And as in these embodiments of unmodified OTS VR/MCD system measurement of the cover-uncover test is through user response, enabling the assessment of misalignments of each eye, nothing eye movements, by VR covering of one eye, recording responses of the uncovered eye, and then quick cover/uncover shifts to note any abnormal compensation eye movements.

In one embodiment the VR target may flicker or change in appearance (i.e., change color) briefly and simultaneously with the blackout of the image in the other eye. Test subject 8 may then provide user response, noting when he or she sees the change of the virtual object. In the normal condition, the eye will already be centered on the target object and will be able to see these brief VR changes in appearance. In conditions where test subject 8 eyes are not aligned, there will be a lag time while test subject 8 re-centers their eye(s) on the target object and may not see the brief change in appearance resulting in an error of omission of the expected response.

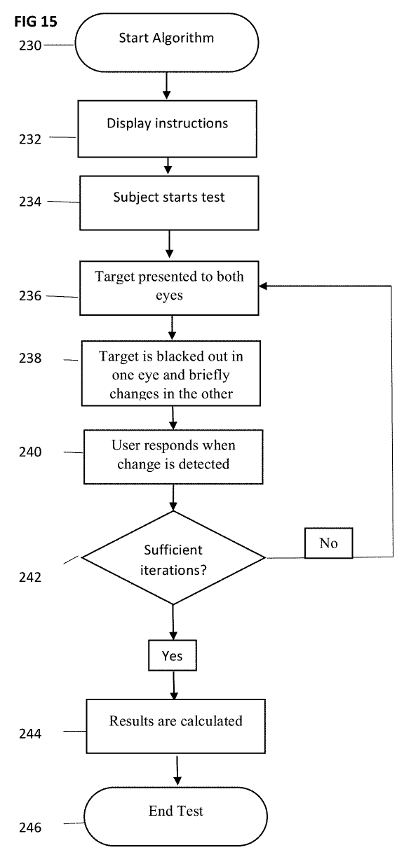

FIG. 15 is a high-level flowchart illustrating a method of use for various for various cover uncover assessments. The cover uncover assessments begins 230, instructions to test subject 8 are displayed on unmodified OTS VR/MCD screen, and may include instructions describing the test, and how test subject 8 is to respond (as listed above) 232. Then, in the user initiated mode, test subject 8 starts the test by a tap of the control pad or remote button press 234. Test subject 8 is presented with the VR images presented in the near vision VR environment where the target image is presented in the same location for both eyes 236. One VR image to one of the eyes is replaced with a completely dark screen, or the target image may simply be removed from presentation in one eye 238, as noted above, and the VR target may change in appearance briefly and simultaneously with the blackout of the image in the other eye 240. And again, as noted, through user response, test subject 8 responds, when a change in the target object is detected 242. Correct and incorrect responses and reaction times are recorded and stored, and the test is repeated for all orthogonal points of interest in the VR visual fields 242 to 236. And responses and test results are stored in the VR/MCD device 246, and/or transmitted wirelessly to computing device 12 for off-line storage and/or further processing.

Color Blindness Screening

In one embodiment of color blindness screening, images are presented in the near vision VR environment in a manner that requires the subject to distinguish between different colors or patterns of colors. The user's ability to discriminate between various colors is measured by user response, as defined above.

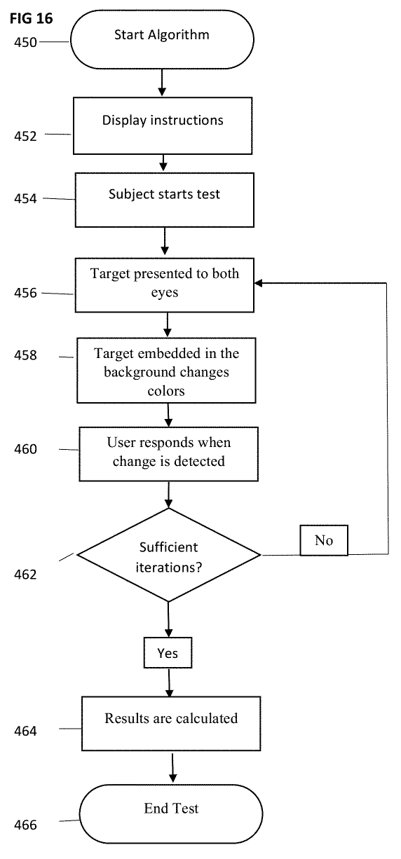

FIG. 16 discloses a high-level flowchart illustrating a method for various for color blindness assessments. The color blindness test begins 450, and instructions to the subject may be displayed on the unmodified OTS VR/MCD screen. These instructions include a description of tests, and how the subject is to respond, as in the above tests 452. In the user initiated mode, test subject 8 initiates the start of the test, as in the above test subject 8 initiated tests, 454, and thereafter test subject 8 is intermittently presented with the VR image of a target object embedded in the virtual environment 456. The color of the object changes with each presentation, sometimes indistinguishable from the background in individuals with color blindness 458 and test subject 8 is instructed as to the proper user response when the target object is detected 460.

Correct and incorrect responses and reaction times are recorded and stored within the unmodified OTS VR/MCD system 462 and/or transmitted wirelessly to computing device 12 for off-line storage and/or further processing 464.

Modified OTS VR/MCD Eye Tracking Systems with Ocular Cameras

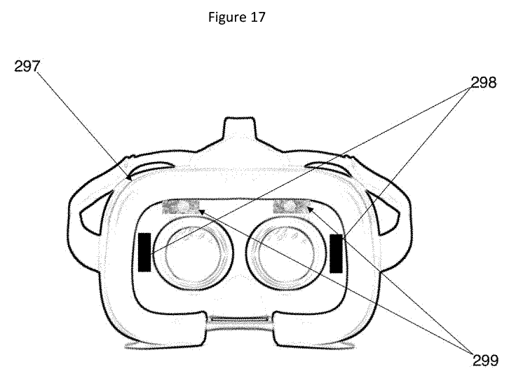

FIG. 17 is an inside view of a modified OTS VR/MCD headset 297 with either embedded cameras 299 or window openings 298 to use the forward-facing cameras of the MCD for more precise eye tracking. These eye movement tracking embodiments modify the OTS VR/MCD headset for ocular camera (OC) for visualizing and recording eye movements/eye tracking. That is, modifying the OTS MCD headset with window openings 298 to use of the internal forward-facing cameras of the MCD as OCs, i.e., as internal ocular cameras (IOC), or embedded ocular eye (EOC) 299 tracking cameras in the headset. In these embodiments, OC eye tracking data algorithms collect and process actual eye movements during the above listed test instead of using user response inputs for the assessments. Direct eye tracking provides more precise responses, thus detecting with greater accuracy various eye movements disorders, such as nystagmus, saccadic movements and pursuit movements in the eye tracking response to tracking stimuli presented in the VR display. Also, other types of eye movements responses, such as convergence, optokinetic nystagmus and resting nystagmus provide a wide variety of neuro-ophthalmology protocols, are within the disclosed eye tracking embodiments, e.g., abnormal eye movements are detected and reported, along with pupil size and reactivity (pupillometry) testing.

Software Algorithm Logic

Software algorithm logic determines the appropriate measures for the task:

Convergence test: what is the virtual distance from the eye when vergence breaks (eyeball position diverges from the fixation point as measured by the camera) and what is the virtual distance when fusion occurs (eyeball position converges to the fixation point as measured by the camera).

Visual fields: an embodiment includes instructing the user to target the virtual fixation point as rapidly as possible with their eyes. Omission of targeting response, overshooting or undershooting of targets are measured, and reaction time from presentation to eye traversing are measured by the camera image analysis.

Extra ocular motility: camera detects whether both eyes are on the fixation target in all direction of gaze. The orientation of each eye is determined in all direction of gaze. The software logic determines which eye muscles/Cranial Nerves are weak based on the orientation of each eye in the directions of gaze. Saccades (rapid eye movements) and nystagmus including optokinetic nystagmus (repetitive bobbing movements) are measured in response to multiple targets presented in the VR environment.

VOR: eye movements are recorded in response to the movement or position of the head. Under compensation or overcompensation of the eye movements in relation to the head movements is measured by camera image analysis.

Ocular tracking: cameras record the position of the eye(s). The accuracy and smoothness of the eyeball pursuit including measures of deviations from the smooth pursuit of the target.

Cover-uncover. The position of the eye(s) is measured before and after each cover. The software logic determines the direction of correction movement upon cover-uncover and correlates this with weakness of the eye muscles/extra-ocular cranial nerves.

Additionally, both the IOC and EOC work in combinations with electrooculography (EOG) embodiments disclosed hereafter (i.e., EOG embodiments with IOC embodiments, and EOG embodiments with EOC embodiments).

Modified OTS VR/MCD for EOG Systems and Methods

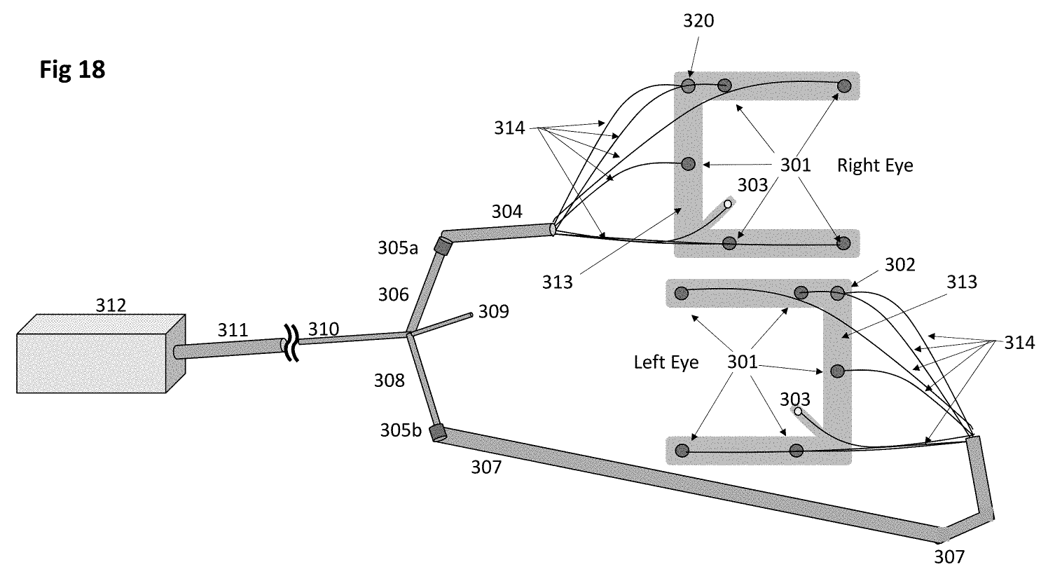

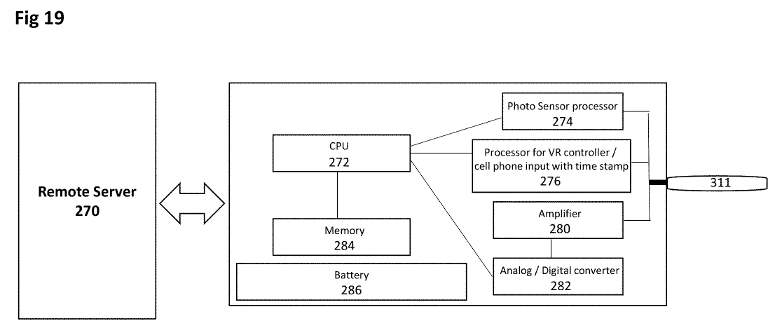

EOG embodiments record changes of the electric field of the eyes generated by movement of each eye independently, by a multiplicity of electrodes placed around each eye. FIG. 18 is a diagrammatic view of a representative embodiment of EOG electrodes 301, plus embedded photo sensors 303 for each eye. Thus, EOG system embodiments consists of a multiplicity of electrodes surrounding the eyes 301 to record the EOG signal. Electrodes 301 are held by an adhesive material 313 that anchors the electrodes onto the skin. A ground electrode 302 is similarly held on the adhesive material 313 of each eye. A photo sensor 303 is incorporated into the adhesive material of each eye electrode. The wires from the eye electrodes, ground electrode and photo-sensor 314 come together into a right and left composite sensor cable 304, 307. Composite sensor cables 304, 307 connect to right and left recorder sensor cables 306, 308 through a right and left connectors 305a, 305b. Right and left recorder sensor cables join, along with the cable to the VR/MCD interface providing time-stamped VR data 309, to form cable 310, which then join and form a main cable 311 to EOG recording unit and data processor 312. This EOG recording unit and data processor 312, in FIG. 18 is the EOG recording unit and data processor disclosed in FIG. 19 (i.e., the entire right side of FIG. 19, excluding remote server 270).

Proceeding to embodiments of the EOG recording unit and data processor of FIG. 19. Main cable to the EOG system 311 contains the wires from the EOG electrodes, photo sensor, and output from the VR display unit, and transmitting all to the EOG Recording Unit of FIG. 18 for processing and storage. Those EOG electrodes, photo sensor, and VR display wires within main cable 311 take separate paths in the EOG recording unit of FIG. 18. EOG wires go the amplifier 315. Photo sensor wires go to the photo sensor processor 317, and wires from the VR display, with time-stamped VR display imaging information, is transmitted to processor for VR controller/MCD input with time stamp 276.

EOG signals are amplified 280 and then digitized by an A/D converter 282, and both are transferred to the CPU 272 and stored in a multiplexed format in memory 284. This data is sampled at rates not lower than 256 Hz from each channel. Amplifier 280 records EOG signals from each eye using a common reference electrode from each eye, allowing reformatting of EOG data with a variety of analysis montages, providing wider analysis possibilities, and not locking the analysis into a single montage format. Filters on the amplifier 280 are set with a suitable very low high pass filter, allowing capture of slow eye movements and a suitable high, low pass filter, allowing capture of fast eye movements. Photo-sensor data from the photo-sensor processor 274 also goes to the CPU 272 and is placed in synch with the multiplexed EOG data to precisely mark when the visual display synchronizing flashes occur. Additionally, time-stamped VR data is transmitted to processor VR controller/MCD 276 for its processing. The system computes any offsets between the time clock of the VR display and the true presentation of the flash as sensed by the photo-sensor, and photo-sensor processor 274.

Embodiments of EOG recording unit and data processor unit 312 (which is the right side of FIG. 19) have multiple placements, e.g., strapped onto the VR headset, or built within the VR headset.

FIG. 21 discloses a representative testing sequence of the combined electrodes of FIG. 18 and its data processing in FIG. 19 used with a suitable VR headset. The EOG electrode set is placed on test subject 8 and secured 250, the modified OTS VR/MCD head set is placed on the subject and right and left composite sensory cables 304, 307 are connected to the right and left cable connectors 305a, 305b, thus connecting the modified OTS VR/MCD head set with FIG. 18 EOG electrodes 301 and embedded photo sensors 303 to the EOG recording unit and data processor of FIG. 19.

EOG recording unit performs a system check, testing electrode impedances and overall signal integrity, and sends the results of the systems check back to the VR display through the connector 209 or wirelessly, step 252. After a successful systems check, test subject 8 selects which test to perform 254, or test selection is controllable by another individual who will have remote access to—controlling the device, through, among other ways, a wireless connection. Thereafter, a synchronization process begins 255 with the transient flash sequence presented for each loop of testing, i.e., a flash sequence per session, at the beginning of each and used as a synchronization between the VR display and the EOG system (as disclosed above in the interaction between FIG. 18 EOG electrodes and their processing in FIG. 19).

After synchronization, the EOG is calibrated by having test subject 8 look at eight cardinal positions, following the position of a dot on the VR display while EOG data is collected. Those positions of eye gaze are: up, down, right, left, right upper corner, left upper corner, right lower corner, left lower corner, and the system creased calibration curses with this data 256 to allow for localizing eye position from the EOG data.

Then a specifically selected eye EOG test sequence begins 257, during which both the VR display goes through the programmed sequence of display changes associated with the task 258 and the EOG system monitors and collects the data 259. This continues until the end of that particular test sequence 260. EOG data is analyzed 261, and eye movement performance is determined, coordinated with the VR imaging on the VR display. Test results can then be sent to the VR display for viewing by test subject 8, and/or also to a remote server wirelessly 262. If sent to test subject 8, he or she can then be given options to end the testing, or return to perform another test 263.

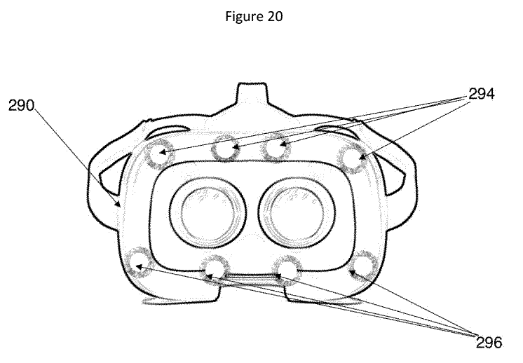

Finally, additional EOG embodiments include embedding EOG sensors in a modified VR/MCD headset. FIG. 20 discloses representative embodiments of an inside view of a modified OTS VR/MCD headset with embedded EOG sensors modified with embedded cameras 294, 295.

The above representative protocols/tests and training scenarios are representative of methods of using the disclosed system modified and unmodified OTS VR/MCD system embodiments and are not intended as a comprehensive list of those assessments and uses envisioned by embodiments of the invention.

Although the invention has been shown and described with respect to a certain preferred systems embodiments and methods of using those systems, it is obvious that equivalent alterations and modifications will occur to others skilled in the art upon the reading and understanding of this specification and the annexed drawings. In particular regard to the various functions within each method of use, or described system elements that are not structurally equivalent to the disclosed structures but performs its function, they are included because of the illustrated exemplary embodiment or embodiments disclosed herein of the invention.

In addition, while a particular feature of the invention may have been described above with respect to only one or more of several illustrated embodiments, such feature may be combined with one or more other features of the other embodiments, as may be desired and advantageous for a particular application.

We claim:

1. A headset having an unmodified combination of (1) an off-the-shelf mobile computing device, and (2) an off-the-shelf virtual reality device, both having a suitable interconnection and software configured for neuro-ophthalmology, vestibular, ocular and oculomotor assessments, said system configuration comprising:
(a) the mobile computing device having
(i) storage for programable results of computing,
(ii) programing virtual reality imaging for the off-the-shelf virtual reality device,
(b) said system capable of communicating spatiotemporal locations of test subject head movements;
(c) said system communicating spatiotemporal coordination between the virtual reality imaging and the spatial sensor data;
(d) said system capable of providing user responses to the system, and
(e) computing said data and responses of test subject head movements in the mobile computing device storing, displaying and transmitting those computations for neuro-ophthalmology, vestibular, ocular and oculomotor assessments.

2. A headset having a modified combination of (1) an off-the-shelf mobile computing device, and (2) a virtual reality device, both having a suitable interconnection and software configured for neuro-ophthalmology, vestibular, ocular and oculomotor assessments, said system configuration comprising:
(a) the mobile computing device having
(i) storage for programable results of computing,
(ii) programing virtual reality imaging for the off-the-shelf virtual reality device,
(iii) internal cameras;
(b) said modified system for using cameras of the mobile computing device to collect and process eye movements for neuro-ophthalmology, vestibular, ocular and oculomotor assessments.
(c) The system of claim 2 further comprising window openings modifications in said headset for use of an internal forward-facing cameras of the mobile computing device.
(d) A fully integrated unit with features a, b, and c

3. A headset having a modified combination of (1) an off-the-shelf mobile computing device, and (2) a virtual reality device, both having a suitable interconnection and software configured for reading electrooculogram signals:
(a) the mobile computing device having
(i) storage for programable results of computing,
(ii) programing virtual reality imaging for the off-the-shelf virtual reality device;
(b) said modified system capable of communicating spatiotemporal coordination between the virtual reality imaging and the spatial sensor data;
(c) a set of multiple electrooculography-electrodes for each eye;
(d) an electrooculogram recording unit connected to the electrode set of (c) amplifying and filtering the electrooculogram signals and thereby providing precise electrooculogram assessments.
(e) The system of claim 4 further comprising at least one embedded photo sensor in the electrodes of (c), and processing photo sensor data by coordinating the photo sensor data with the spatiotemporal virtual reality imaging,
(f) A fully integrated unit with features a, b, c, d, and e

4. A method for using the system of claim 1 for balance assessments, the method comprising the steps of:
presenting various static and dynamic virtual reality imaging in the system of claim 1;
collecting spatial sensor data from the mobile computing device having spatial sensor data during the balance test;
analyzing test subject spatial movement from collected mobile computing device spatial sensor data;
providing balance assessments based of analyzed spatial sensor data per various balance protocols.

5. A method for using the system of claims 2, 3, and 4 for neuro-ophthalmologic and vestibulo-ocular assessments, the method comprising the steps of: presenting visual stimuli in the virtual reality environment assessing convergence, visual fields, extraocular motility, ocular tracking, vestibular-ocular reflex, cover-uncover testing, and color blindness using the off-the-shelf or modified versions with extraoculogram capability, on-board cameras of the mobile computing device or embedded cameras of the invention or any combination these unmodified or modified embodiments.
